(19) United States
(12) Patent Application Publication
(10) Pub. No.: US 2026/0224166 A1
(43) Pub. Date: Aug. 6, 2026

(54) A NONINVASIVE WEARABLE FOR THE PHYSIOLOGICAL ASSESSMENT OF PHYSICAL AND COGNITIVE READINESS FOR MILITARY TASKS

(71) Applicant: EMERJA CORPORATION, Monte Sereno, CA (US)

(72) Inventors: Guy Miller, Monte Sereno, CA (US); Daniel Hansen, Santa Cruz, CA (US)

(21) Appl. No.: 19/099,207

(22) PCT Filed: Aug. 10, 2023
(86) PCT No.: PCT/US2023/072033

§ 371 Date: Jan. 28, 2025

Related U.S. Application Data
(60) Provisional application No. 63/371,365, filed on Aug. 12, 2022.

(51) Int. Cl. A61B 5/00; A61B 5/01; A61B 5/03; A61B 5/107

(52) U.S. Cl. CPC A61B 5/6843; A61B 5/01; A61B 5/03; A61B 5/1073; A61B 5/6823; A61B 5/6831; A61B 2562/0247

(57) ABSTRACT

Disclosed is a device, wearable by a user, comprising a band configured to be worn in substantially complete cross-sectional contact with a biological compartment of the user; a tension mechanism connected to the band, the tension mechanism configured to allow a circumference of the band (i) to expand to accommodate ingress of fluid into the biological compartment and (ii) to contract to accommodate egress of fluid out of the biological compartment, such that the band maintains substantially complete cross-sectional contact with the biological compartment of the user; and a sensor connected to the band, the sensor configured to collect a plurality of patient data. The device is suitable for use in assessing military readiness of an individual or a cohort of individuals.

### INCORPORATION BY REFERENCE TO RELATED APPLICATIONS

Any and all priority claims identified in the Application Data Sheet, or any correction thereto, are hereby incorporated by reference under 37 CFR 1.57. This application is the national phase under 35 U.S.C. § 371 of prior PCT International Application No. PCT/US2023/072033 which has an International Filing Date of Aug. 10, 2023, which designates the United States of America, and which claims priority to U.S. Provisional Application No. 63/371,365, filed Aug. 12, 2022. Each of the aforementioned applications is incorporated by reference herein in its entirety, and each is hereby expressly made a part of this specification.

### BACKGROUND

### Field of the Disclosed Technology

The disclosed technology generally relates to systems for, wearable devices for, and methods of obtaining measurements of emergent properties of a complex adaptive system, such as a biological system, an organism, or for example a human, or a non-biological system. More specifically, the disclosed technology relates to a noninvasive wearable device for the physiological assessment of physical and cognitive readiness for military tasks. The disclosed technology also generally relates to an automatically adjusting or manually adjustable band.

### DESCRIPTION OF THE RELATED ART

A historic perspective sheds light on the manifold advantages of the disclosed technology⁷. Known devices for and methods of monitoring biological systems, and known systems for maintaining or improving health are inadequate. For example, known wearable devices generally exist in the off-the-shelf market; however, many patients and users are excluded from that market. Furthermore, and as a further example, known wearable devices are generally “one size fits all”; however, many patients and users cannot be accommodated by such devices. Additionally, wearable devices generally will accomplish their intended functions only when worn as intended and serve little or no function if not worn substantially continually. There are usually critical moments in which the wearable device should be worn, but off-the-shelf market and one size fits all devices may be unworn at those moments and have features that often motivate a patients and users to remove the wearable device.

### SUMMARY

Designs of, method for making, methods of adjusting, and methods of improving compliant use and extended use of an adjustable wearable device are disclosed. The disclosed technology provides a device that is wearable by a user, the device comprising a band configured to be worn in substantially complete cross-sectional contact with a biological compartment of the user; a tension mechanism connected to the band, the tension mechanism configured to allow a circumference of the band (i) to expand to accommodate ingress of fluid into the biological compartment and (ii) to contract to accommodate egress of fluid out of the biological compartment, such that the band maintains substantially complete cross-sectional contact with the biological compartment of the user; and a sensor connected to the band, the sensor configured to collect a plurality of patient data. Method of collecting and distributing data, collected via the device, are also disclosed. In certain embodiments of the devices and of the methods disclosed, the data relates to the health capacity of the user. In certain embodiments of the devices and of the methods disclosed, the data relates to the heat flux of the user. In certain embodiments of the devices and of the methods disclosed, the data relates to the heat flux of the user over multiple circadian cycles.

The disclosure provides designs and methods of manufacturing a device for continuously and contextually characterizing an individual's metabolic state by measuring their thermal signature to assess what is referred to as a thermoregulatory phenotype. Changes relative to this phenotype are sensitive indicators of change in health state. The device is designed and configured such that it delivers, in human use, general associations between an individual's thermal signature and physiologic reserve. Additional information is available within the thermal signature to an actionable assessment of readiness. Clinical studies are designed to gather data that will serve as a novel vital sign of homeostasis as well as an aggregate health signature, with applicability to early detection of many disease states, management of individual wellness and readiness.

In certain embodiments, the disclosed technology provides a device wherein the tension mechanism is directly connected to the sensor.

In certain embodiments, the disclosed technology provides a device wherein the tension mechanism is directly connected to the band.

In certain embodiments, the disclosed technology provides a device wherein the tension mechanism is located within a clasp that is directly connected to the band.

In certain embodiments, the disclosed technology provides a device wherein the tension mechanism includes a first mechanism and a second mechanism located in parallel when the band is in a closed position.

In certain embodiments, the disclosed technology provides a device wherein the clasp is located in parallel with the sensor when the band is in a closed position.

In certain embodiments, the disclosed technology provides a device wherein a first end of the band is configured to wind into a first band adaptor located within the tension mechanism, and a second end of the band is configured to wind into a second band adaptor located within the tension mechanism, wherein the first band adaptor and the second band adaptor configured to stabilize the first end of the band and the second end of the band.

In certain embodiments, the disclosed technology provides a device wherein the tension mechanism comprises a spring.

In certain embodiments, the disclosed technology provides a device wherein the spring is embedded within the band.

In certain embodiments, the disclosed technology provides a device wherein the tension mechanism comprises a slider.

In certain embodiments, the disclosed technology provides a device wherein a first end of the band overlaps with a second end of the band.

In certain embodiments, the disclosed technology provides a device wherein the tension mechanism is round.

In certain embodiments, the disclosed technology provides a device wherein the tension mechanism is knurled.

In certain embodiments, the disclosed technology provides a device wherein the tension mechanism is configured to bring the first end of the band and the second end of the band together or apart.

In certain embodiments, the disclosed technology provides a device wherein the clasp further includes at least two pulleys, the at least two pulleys configured to transmit tension from the band.

In certain embodiments, the disclosed technology provides a device wherein a first connector is placed on a first pulley of the at least two pulleys and includes a first connector end and a second connector end, the first connector end is fixed, the second connector end is connected to the tension mechanism, the first connector is parallel to a second connector.

In certain embodiments, the disclosed technology provides a device wherein a second connector is placed on a second pulley of the at least two pulleys and includes a first connector end and a second connector end, the first connector end is fixed, the second connector end is connected to the tension mechanism, the second connector is parallel to a first connector.

In certain embodiments, the disclosed technology provides a device wherein the tension mechanism is configured to adjust based on a circumference of the biological compartment of the user.

In certain embodiments, the disclosed technology provides a device wherein the tension mechanism is configured to adjust automatically based on the circumference of the biological compartment of the user.

In certain embodiments, the disclosed technology provides a device wherein the biological compartment is at least one of a wrist and an ankle.

In certain embodiments, the disclosed technology provides a device wherein the biological compartment is at least one of a finger and a toe.

In certain embodiments, the disclosed technology provides a device wherein the biological compartment is at least one of a lower leg and a forearm.

In certain embodiments, the disclosed technology provides a device wherein the biological compartment is at least one of a thigh and an upper arm.

In certain embodiments, the disclosed technology provides a device wherein the biological compartment is an abdomen.

In certain embodiments, the disclosed technology provides a device wherein the sensor is configured to dynamically measure pressure of the biological compartment.

In certain embodiments, the disclosed technology provides a device wherein the sensor is configured to dynamically measure the circumference of the band.

In certain embodiments, the disclosed technology provides a device wherein the sensor is configured to dynamically measure heat flux of the biological compartment.

In certain embodiments, the disclosed technology provides a device wherein the sensor is configured to dynamically measure volume of the biological compartment.

In certain embodiments, the disclosed technology provides a device comprising an additional senor, wherein the additional sensor is configured to dynamically measure tension of the band.

In certain embodiments, the disclosed technology provides a device comprising an additional sensor, wherein the additional sensor is configured to dynamically measure tension of the tension mechanism.

In certain embodiments, the sensor is configured to remain flush to the biological compartment of the user as the tension mechanism allows the circumference of the band to expand and contract.

In certain embodiments, the disclosed technology provides a method of adjusting a device that is wearable by a user, the method comprising: placing a band on a biological compartment of a user, the band configured to be worn in substantially complete cross-sectional contact with a biological compartment of the user; and winding a tension mechanism that is configured to receive a first end of the band and a second end of the band, the tension mechanism configured to bring the first end of the band and the second end of the band together or apart.

In certain embodiments, the disclosed technology provides a method wherein the band is further configured to be worn in substantially complete cross-sectional contact with a biological compartment of the user based on a comfort level of the user.

In certain embodiments, the disclosed technology' provides a method wherein winding the tension mechanism presses a sensor connected to the band into the biological compartment of the user, the sensor configured to collect a plurality' of user data.

In certain embodiments, the disclosed technology provides a method wherein winding the tension mechanism occurs automatically based on a circumference of the biological compartment.

In certain embodiments, the disclosed technology' provides a method for collecting data from a device that is wearable by a user, the method comprising: dynamically adjusting a tension mechanism connected to a wearable based on a circumference of a biological compartment of the user; dynamically sensing at least one emergent factor of a biological system of the user; and generating a plurality of data relating to the at least one emergent factor.

In certain embodiments, the disclosed technology provides a method wherein the plurality of data comprises surface temperature and physical activity of the biological system over time.

In certain embodiments, the disclosed technology provides a method further comprising estimating heat elimination of the biological system over time based on differential surface temperature; estimating heat production of the biological system over time based on physical activity'; and estimating a basal metabolic status of the biological system based on temporal alignment of heat elimination and heat production.

In certain embodiments, the disclosed technology provides a method further comprising obtaining a quasiperiodic rhythm of the biological system based on the plurality of data, wherein the quasiperiodic rhythm is of seconds-timescale, of minutes-timescale, ultradian, circadian, circalunar, or of yearly timescale.

In certain embodiments, the disclosed technology' provides a method further comprising obtaining a variability of the quasiperiodic rhythm across a predetermined amount of time; and determining a health capacity' based on the variability of the quasiperiodic rhythm.

In certain embodiments, the disclosed technology' provides a method further comprising estimating heat elimination of the biological system over time based on differential surface temperature; estimating heat production of the biological system over time based on physical activity; estimating a basal metabolic status of the biological system based on temporal alignment of heat elimination and heat production; and determining a health capacity by applying a time-dependent function to the estimated basal metabolic status, wherein the time-dependent function is derived from the quasiperiodic rhythm of the biological system.

In certain embodiments, the disclosed technology provides a method wherein the plurality' of data comprises heat flux data.

In certain embodiments, the disclosed technology provides a method wherein at least one health capacity is a basal metabolic status, and at least one emergent factor is a temporal alignment of heat production and heat elimination.

In certain embodiments, the disclosed technology' provides a method wherein the temporal alignment is related to at least one quasiperiodic rhythm of the biological system.

In certain embodiments, the disclosed technology provides a method wherein the at least one quasiperiodic rhythm is a circadian rhythm.

### BRIEF DESCRIPTION OF THE DRAWINGS

FIGS. 1A, IB, 1C, 2A, 2B. and 2C illustrates certain wearable devices known in the art.

FIGS. 3A and 3B illustrate an example embodiment of a tension mechanism.

FIG. 4 illustrates an example embodiment of a tension mechanism.

FIGS. 5A and 5B illustrate an example embodiment of a tension mechanism within a holder.

FIGS. 6A and 6B illustrate an example embodiment of an internal compartment of a tension mechanism.

FIG. 7 illustrates an example embodiment of a tension mechanism.

FIG. 8 illustrates an example embodiment of a band with a clasp.

FIGS. 9A and 9B illustrate an example embodiment of a tension mechanism.

FIG. 10-17 illustrates an example embodiment of a wearable device.

FIG. 18 illustrates an example embodiment of a sensor.

FIG. 19 illustrates an example embodiment of a sensor.

FIG. 20 illustrates an example embodiment of a sensor.

FIG. 21 illustrates an example embodiment of a sensor.

FIG. 22-23 illustrates an example embodiment of a battery.

FIG. 24-25 illustrates an example embodiment of a thermal disc.

### DETAILED DESCRIPTION OF THE PREFERRED EMBODIMENT

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part incorporated herein by reference in their entireties for the purposes indicated by the context of their citation herein. However, the citation of any document is not to be construed as an admission that it is prior art with respect to the present disclosure.

The generation, analysis, and use of data relating to the health capacity of biological systems has been explored. More specifically, the use of sensors, and combinations of sensors, for capturing data related to the health capacity of biological systems has also been explored. Health capacity can refer to the resilience (adaptivity) of a system expressed primarily by its ability to persist or achieve some core function. To assess the health capacity⁷ of a system, the one may interpret the emergent factors of the system. Emergent factors can refer to the properties of water or of an aqueous system or of a biological system or of a complex adaptive system that may be related to, or form the basis for assessing, the health capacity⁷ of the system. Emergent factors may also refer to events, deviations from norm or other time dependent patterns in some measurable parameter of the system that can be observed directly or indirectly. Emergent factors or properties may also refer to properties of a biological system that are not readily predictable from the functions of the component parts of the system. Examples of emergent properties can include amphotericity, conductivity, solvation capacity, ion mobility, oxidation-reduction potential, ligan association, hydration, electrolysis, thermal conductivity, heat capacity, thermal absorptivity, adhesion, cohesion, transparency, turbidity, incompressibility, polarity, dipolarity, dipole movement, diamagnetism, voltage range of the liquid phase, temperature range of the liquid phase, abundancy, and speciation, flux of energy, momentum, particles or other substances, heat elimination, either as an absolute, statis value of heat elimination or as a periodic function, for example, a circadian periodicity of heat elimination.

As water is central to the function of every chemical or physical process in all biological systems at all scales (For example, whole body, cell, tissue, organ, etc.) and the availability of sufficient associated water and any of the aforementioned entities associated with water, either alone or in combination, is thus important to the function of a biological system it is therefore useful to select and utilize sensors which measure directly and/or indirectly the properties of water and any of the aforementioned entities associated with water, either alone or in combination, for quantifying and learning at least the following operational properties of a biological system. For example:

High Heat Capacity: The relatively high heat capacity of water and aqueous systems provides thermal stability to the milieu interior of a biological system. In contrast, other typical or abundant solvents, have substantially less than half the heat capacity of water.

Incompressibility: The relative incompressibility' of water and aqueous systems have anomalously high thermal diffusivity, comparable to that of a solid. In contrast, other typical or abundant solvents are substantially more compressible and therefore susceptible to structural damage.

Large Thermal Diffusivity: Water and aqueous systems have anomalously high thermal diffusivity', comparable to that of a solid. This enables the deleterious internal temperature variations around active organelles to be minimal. In contrast, other typical or abundant solvents are substantially less efficient at distributing energy and would have much larger temperature gradients around metabolizing centers potentially resulting in structural degradation.

Large Infrared Absorption Band: Metabolism makes and breaks carbon bonds in specific ways to build organic structures. The waste heat from these processes is efficiently captures by the infrared absorption band of water. In contrast, other typical or abundant solvents are substantially less efficient at capturing heat. Efficient capture of heat is also essential to rapid enzyme kinetics.

Currently there is not an agreed upon measure of health. Health is frequently defined as the absence of disease (symptoms). Disease metrics are lagging indicators of failing health, and thus do not reflect health in an affirmative sense, and are not in and of themselves optimizable vis-a-vis real health, not disease, outcomes. Advances in the understanding of disease have revealed that early changes in inflammation may be predictors of disease, but inflammation too is a late sign of pathogenesis.

Inflammation is a common pathway that can affect every organ system in the body. The inflammatory' response can be triggered by an array of stimuli or stresses ranging from a normal response to exercise and training, to mechanisms now associated with oncogenesis and neurodegeneration. The clinical signs of inflammation have been classically defined as the pentad of: increased heat, pain, redness, swelling, and loss of function, and now early inflammation-so called pre-inflammation, is a risk factor for disease. We note the association between changes in water, and emergent parameter, and inflammation, e.g., temperature and swelling.

Edema is one example of inflammation. Edema can occur when fluid builds up in at patient's tissues and can affect anyone by causing parts of the patient's body to increase in size. For example, the circumference of a patient's biological compartment may increase in size. As explained above, edema can be a symptom of an underlying health condition. The underlying health condition can be monitored or assessed by use of a wearable device that can collect and/or monitor the health capacity of biological systems. It may include at least one wearable thermodynamic sensor that can be configured to measure an emergent factor of the human, wherein the emergent factor is the temporal alignment of heat production and heat elimination of the human, the temporal alignment relating to the circadian rhythm of the human, and based on the emergent factor, generate measured data comprising heat flux data over time. The wearable device may also capture heat flux data, wherein at least one health capacity⁷ is a basal metabolic status, and at least one emergent factor is the temporal alignment of heat production and heat elimination of the biological system. The wearable device may include an array of sensors that record health metrics and capture the data. The wearable device may continuously record select “energy signatures” metrics or indicators of health for the subject. In some embodiments, the wearable device requires low cost and low power, enabling accessibility and continuous data capture in real-time. In some embodiments, the wearable device comprises a multimodality sensor system that measures electrochemical, mechanical, structural, thermal, and/or energetic properties reflective of homeostasis and cell physiology. The wearable device can comprise any number of sensors.

Mild or severe cases of edema can cause the patient to be unable to wear the wearable device that would be able to monitor and/or assess the health capacity of the patient's biological compartment. The patient may remove the wearable device because it is too tight because the wearable device may be constricting the patient's biological compartment. In some cases, a severe case of edema may not allow the patient to use any already available wearable device because a size that would accommodate the patient's edema is not available. The data related to the health capacity of biological systems cannot be captured or assessed unless the patient is continuously wearing the wearable device that allows the capture and assessment of the data. In other words, the stream of data collected by the wearable device is terminated upon the user removing the wearable device.

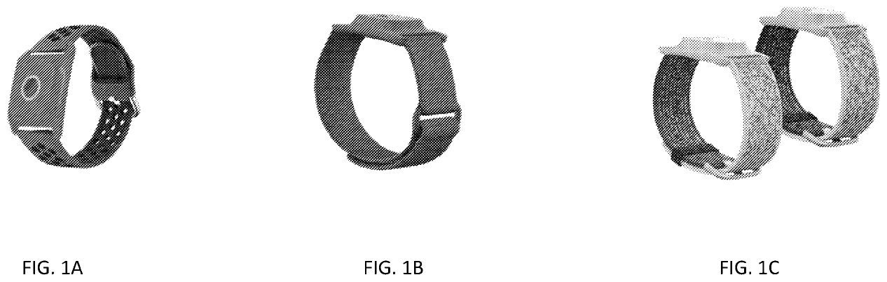

Certain wearable devices know n in the art, as depicted in FIGS. 1A, IB, 1C, 2A, 2B, and 2C, include a variety of passive and active adjustable wearable devices that provide an “off the shelf’ solution. Active adjustable wearable devices include band styles that require the user to manually adjust the size of the band. These adjustments can be macro or micro adjustments. For example, the band may be manufactured in different sizes with a flexible material. For example, the band may be a size “SMALL,” which may fit a user's wrist 5.5″-7″ in circumference, or “LARGE,” which may fit a user's wrist 7.1″-8.7″ in circumference. The limited range in adjustability may lead to uncomfortable wear for the user, which may cause the user to remove the device, thereby terminating any stream of data.

Therefore, there exists a need for devices that allow for optimal monitoring health capacity and heat flux. Disclosed herein is such a device. In some embodiments, the wearable device may include a band, a tension mechanism, and a sensor. The wearable device may be worn around a biological compartment to measure an emergent factor. As used herein, the biological compartment can refer to a wrist, ankle, finger, toe, lower leg, forearm, thigh, upper arm, and/or abdomen. The biological compartment may undergo a change in homeostasis that may cause the size or shape of the biological compartment to change or vary. The biological compartment may undergo a change in homeostasis in which the flow of water is anything but normal. For example, a patient with any element of disease may experience or undergo a dramatic change in the diameter of a biological compartment. It is during a change in homeostasis of the biological compartment where the monitoring of a patient's health capacity may not be optimal. In some embodiments, the wearable device may adapt to the change in homeostasis exhibited by the biological compartment being measured by the wearable device.

In some embodiments, the band may be fitted for a user. For example, the band may vary in size (e g., small, medium, large) to allow the band to be worn by users with various size biological compartments. In some embodiments, the band be adjustable and may include a flexible material. Such flexible material may include nylon, rubber, silicone, fabric, etc. In some embodiments, the adjustability of the band may allow a patient to continue to wear the wearable device while the biological compartment is undergoing or experiencing a change in homeostasis. For example, the circumference or length of the band may increase or decrease. In other words, the circumference or length of the band can adjust to accommodate or fit the circumference of the biological compartment. This may allow the patient to remain comfortable (e.g., not feel restricted) when wearing the device and experiencing a change in homeostasis. Similarly, this may allow the patient to continue to wear the wearable device at all times which can allow the wearable device to collect a constant data stream. The adjustable band can allow patients susceptible to irregular biological compartment homeostasis's to wear the wearable device and monitor their health capacity. Furthermore, in some embodiments, the adjustability of the band allows for the sensor measuring or assessing the various emergent factors to remain in a substantially complete connection with the biological compartment intended to be measured or assessed. This may allow the sensor to maintain a continuous and constant data stream collection because without a substantially complete connection of the sensor to the biological compartment being measured, the sensor cannot assess or monitor the biological compartment.

### Military Applications, Including Assessment of Military Readiness.

Physicists have encountered the problem of emergent orders before (e.g., magnetism) and have concluded that it may be advantageous to identify a thermodynamic parameter which summarizes the order, rather than to attempt to measure molecular details of that order directly. In fact, all order is associated with missing energy. (See, for example, https://en.wikipedia.org/wiki/Latent_heat). For example, when studying complex materials, physicists look for anomalous specific heats as the bellwether of hidden organization. Landau defined the order parameter (See, for example, https://en.wikipedia.org/wiki/Landan_theory): a useful mathematical device which quantifies the thermodynamic character and robustness of the underlying order.

Our insight is based, in part, on the concept that the organization of living systems has associated thermodynamic signatures analogous to order parameters. And, only these biological order parameters will enable highly accurate learning with small sample sizes. Furthermore, it is likely that such a thermal signature may inform us of the robustness of biological order-physiologic reserve and readiness.

A wearable device, which may be referred to as Enerji™, is designed and configured to quantify physiologic energy outputs (for example, peripheral heat and physical activity). This device is benchmarked against gold-standard physiologic endpoints in multiple IRB-approved human studies. Metrics for such benchmarking involve a signal with high accuracy with training sets as small as 25 samples.

A robust structure is identified in human heat signatures and serves as a direct measure of the autonomic processes underlying homeostasis (i.e., biological organization). Specifically, the device provides a means for the non-invasively detection of a thermal signature of an inflammatory' cascade before any change in core temperature. This observation has ramifications from the perspectives of thermal physics, and transformational biological applications.

Despite fundamental differences in the male and female hypothalamic-adrenal-pituitary' axis there is an absence of readily available metrics characterizing the functional capability of the neuroendocrine response essential to the prevention and survival of trauma injury (REF). Instead, it is often assumed that physiological responses to trauma are similar between men and women, with standard metrics defaulting to those of men potentially resulting in sub-optimal treatment of female trauma injuries. The wearable device described herein utilizes a physical model of temperature homeostasis, inspired by the function of the hypothalamus, to interpret the health significance of an individual's thermal signature (See References 1-5). By measuring the principal data streams which the hypothalamus integrates (heat and body temperature), the device allows for the characterizing of the basis of homeostasis and physiologic reserve-including differences between the sexes (See References 4, 5)—and for defining gender-specific metrics that are relevant to trauma injury treatment (See Reference 6). The wearable device continuously and contextually measures these principal data streams moderated by the hypothalamus, and provides a means for characterizing both individuals and gender groups by measuring their thermal signature to assess what we call a thermoregulatory phenotype.

The disclosed technology is based, in part, on the utilization of a novel physical model of temperature homeostasis, providing a means for understanding and/or interpreting the health significance of an individual's thermal signature (thermal phenotype) and for acting upon that interpretation in a variety of ways. The non-invasive wearable device continuously senses thermal signature of body heat, distinct from and superior to simple skin thermometry, and requires no charge or battery replacement for periods as long as several months

The disclosed technology relates to a noninvasive wearable device for the physiological assessment of physical and cognitive readiness for military Tasks. The disclosure provides designs and methods of manufacturing a device for continuously and contextually characterizing an individual's metabolic state by measuring their thermal signature to assess what is referred to as a thermoregulatory phenotype. Changes relative to this phenotype are sensitive indicators of change in health state. The device is designed and configured such that it delivers, in human use, general associations between an individual's thermal signature and physiologic reserve. Additional information is available within the thermal signature to an actionable assessment of readiness. Clinical studies are designed to gather data that will serve as a novel vital sign of homeostasis as well as an aggregate health signature, with applicability to early detection of many disease states, management of individual wellness and readiness.

The readiness of a warfighter is determined by their physiologic reserve, or their capacity to adapt and perform (e.g., maintain homeostasis) under stress. This “reserve” can be assessed clinically by a variety of stress tests but has never been reduced to an accurate physical measurement which can be performed passively by a wearable device. Our goal is to automate the measurement of physiologic reserve using a wearable device so that readiness can be assessed continuously for an individual and at population scale.

Currently, when stress testing is not possible, readiness is quantified by manual assessment of vital signs and other physiologic markers/tests by a medical professional. Because the panel of physiologic data currently available has only weak correlation with physiologic reserve in young healthy subjects, the medical professional must execute a difficult clinical judgement when assessing each patient. As a result, this approach has a large subjective component, limiting both accuracy and scalability. Our approach assumes that readiness and physiologic reserve are determined by the organization of biological energy' resources which underlie homeostasis. While some consumer wearables measure skin temperature, none measure actual energy. Using a novel sensor configuration, the Enerji™ device can estimate peripheral heat elimination, a rich and new measure of physiologic energy which is fundamentally related to homeostasis and hypothalamic regulation. By continuously compiling this signature, we have identified and quantified previously unobserved patterns which we call the thermoregulatory phenotype. Our hypothesis is that these structures are directly determined by autonomic control of thermoregulation and therefore reveal homeostatic stress in real time, thereby enabling prediction of readiness.

The Enerji™ device can deliver on the hardware requirements of this BAA. Our first IRB-approved human trials have shown general associations between our signal and physiologic reserve. The remaining challenge is to distill the general information of our data stream down to an actionable assessment of readiness. Current trials are gathering data towards this goal. If successful, we will have discovered a new vital sign of homeostasis that serves as an aggregate health signature of an individual, with applicability in the assessment of readiness as well as early detection of many disease states, and management of individual wellness.

The innovation, in certain embodiments, utilizes a physical model of temperature homeostasis, inspired by the function of the hypothalamus, to interpret the health significance of an individual's thermal signature (References 1-4). By measuring the principal data streams which the hypothalamus integrates (heat and body temperature), we reveal the logical basis of homeostasis and physiologic reserve. Therefore, instead of using a “‘molecular biomarker” to determine health, our approach uses a homeostatic thermal energy marker. Our prototype wearable device senses this signature continuously, requiring no charge or battery replacement for several months. In a military environment, we envision a closed, secure wireless ecosystem to enable timely data collection and analysis yielding readiness results within several hours of deployment (Figure A). Because we have a physiological sensor and prototype already partially developed, our main technical challenge lies in collecting relevant data which will allow us to establish robust thresholds connecting metrics of an individual warfighters' thermoregulatory phenotype to their grade of physical and cognitive readiness.

Robust models of readiness are developed owing to both (1) the physical connection to homeostasis and hypothalamic function and (2) already collected human study data, as discuss below.

The disclosed technology has the capacity to non-invasively detect inflammation, a common route cause of illness. Specifically, in a yet-unpublished, IRB-approved human subjects' study, healthy volunteers were injected with lipopolysaccharide (LPS) to stimulate inflammation and serial biomarkers of inflammation were measured (See Reference 5). In parallel, subjects' thermoregulatory signatures were non-invasively recorded. The plots in the figure shown below depict a the baseline (in blue) joint distribution of heat and skin temperature over approximately 30 days for three individuals (one per row) from a recent study. The triangular frame is included to highlight inter-subject variability. The study protocol included two injections of LPS separated by a week. The sensor records taken in the hours after first and second LPS injections are indicated in red and green, respectively, as a time-course over a 4-hour period after the injections. The data depicted is raw measurement data without any pre-processing, intended only to the show the structured relationship between skin temperature and body heat which is regulated by the hypothalamus.

FIG. 2: Thermal response to two LPS injections in 3 subjects over 4 hours

Despite inter-subject differences, the results show a common pattern of thermal response prior to the onset of fever, as well as a readily observable post-symptomatic febrile response. The results comport to know n physiology—the hypothalamic control of core temperature (Reference 6). The pre-febrile response involves outlier points on the lower left quadrant (low heat, low skin temperature), a vasoconstricted state which precedes and facilitates the elevation of core body temperature. At later times, we see outlier points in the upper right (high heat, high skin temperature) which correspond to a state of dissipating fever.

This data shows that pre-symptomatic signal of inflammatory stress is detectable by the Eneiji™ device. By measuring warfighters in typical states of stress the technology can map thermal responses onto grades of readiness. By measuring a central parameter of core hypothalamic logic-heat—the disclosed technology can identify stress patterns from a single subject. Indeed, the inflammatory response seen in FIG. 2 is visible in each individual independently. Capturing the inter-subject vanability is not strictly necessary to render the signal useful, though it offers the potential for statistical optimizations.

Model are developed to construct a representative cohort of warfighters and subject them to typical stressors so that we can observe and place thresholds around their characteristic stress responses. This type of study wall be similar in structure and complexify to the we are already running. Basic statistical methods and visualizations similar to FIG. 2 are used to isolate and grade the outlier records associated with states of stress in preparation for the Internal V&V demonstration at the conclusion of Phase 1.

Similar experiments are conducted using tests of cognitive readiness. These experiment show how to determine how stressed thermoregulatory phenotypes relate to cognitive performance. A strong relationship between cognitive function and thermoregulation is shown (See Reference 7-10). The device is also suitable for assessing cognitive readiness.

A challenge of a molecular biomarker is in establishing its significance relative to know n physiology' and homeostasis. Because the disclosed technology⁷ measures body heat, which is fundamentally related to temperature homeostasis, is avoid challenges to the typical molecular biomarker.

An appropriate compute and readout capability is developed. The wearable device can be ruggedized. The above-described data analysis platform is deployable on a public cloud and can use commodity computer, network, and storage services. The architecture may be redesigned to achieve SWAP requirements for in-theater military' use.

Clinical Studies: Conduct such studies to develop the models to stratify the readiness states of ‘“healthy” individuals under various stress loads.

Data Analytics: Augment data analytics to develop military-specific readiness models based on our sensor data and clinical study results.

Hardware: The device is ruggedization to military' specification requirements and selection and/or development of the data collection and display hardwear that achieves operational and SWAP requirements.

Software: Augment software to integrate wearable sensor devices; portable computer and readout capabilities; and centralized capabilities (e.g., data storage, remote upgrade capabilities, etc.) that are specific to department of Defense operational requirements.

In some embodiments, the band may include at least one strap. In some embodiments, the band may include two straps that may be parallel to each other. In some embodiments, the band may include a casing. In some embodiments, the band or casing may be textured. In some embodiments, the patient may insert a portion of the biological compartment through an opening formed by the band when the band is in a closed position. In some embodiments, the band may include a first portion and a second portion. In some embodiments, the first portion may include a receiver that may receive the second portion of the band.

In some embodiments, the band may be connected to a tension mechanism. In some embodiments, the tension mechanism may receive the band. The tension mechanism may include at a first slot that can receive a first portion of the band and a second slot that can receive a second portion of the band. In some embodiments, the tension mechanism may implement a winding function to adjust the band. For example, the tension mechanism may be rotated to wrap the band around the tension mechanism, thereby tightening the band or decreasing the length of the band. Similarly, the tension mechanism may be rotated in an opposite direction to unwrap the band from the tension mechanism, thereby loosening the band or increasing the length of the band. In some embodiments, the tension mechanism may be controlled automatically or manually. In some embodiments, the tension mechanism may be round. In some embodiments, the tension mechanism may be knurled to allow the patient to better grip the tension mechanism.

In some embodiments, the tension mechanism may include a spring. In some embodiments, the spring may be embedded within the band to allow the band to expand. For example, the patient may pull the band, causing the spring to lengthen, which thereby may adjust the length of the band. The band may be at its smallest length or circumference when the spring is at its shortest length.

In some embodiments, a portion of the tension mechanism may be positioned within a clasp that may be directly connected to the band. The clasp may allow the band to remain in a closed position when the patient is wearing the wearable device. In some embodiments, the clasp may include at least one pulley. The pulley may include a channel that receives a middle portion of the band. For example, a first pulley may include a first channel that may receive a first middle portion of a first portion of the band. Similarly, a second pulley may include a second channel that may receive a second middle portion of a second portion of the band. The first and second middle portion of the band may be positioned within the first and second channel of the pulleys, respectively. A second portion of the tension mechanism may be positioned in parallel to the first portion of the tension mechanism. The parallel configuration may form an opening for the patient to insert the biological compartment into. The second portion of the tension mechanism may include a first connection point and a second connection point. The first connection point may be positioned on a first side of the tension mechanism and the second connection point may be positioned on a second side of the tension mechanism. The connection points may serve to connect the band to the tension mechanism. The tension mechanism may further include anchor points to stabilize the band within the tension mechanism. For example, a first end of the first portion of the band may connect to the first connection point and a second end of the first portion of the band may connect to a first anchor point located on the opposite side of the tension mechanism of the first connection point. Similarly, a first end of the second portion of the band may connect to the second connection point and a second end of the second portion of the band may connect to a second anchor point located on the opposite side of the tension mechanism of the second connection point. The tension mechanism may further include knob that may be rotated to tighten or loosen the band. This may cause the band to increase or decrease in circumference.

In some embodiments, the wearable device may include an array of sensors. In some embodiments, the sensors may record health metrics. In some embodiments, the wearable device continuously records select energy signature metrics or indicators of health for the user. In some embodiments, the wearable device captures emergent-derived complexity at the scale of cell physiology. In some embodiments, the wearable device requires low cost and low power, enabling accessibility and continuous data capture in real-time. In some embodiments, the wearable device comprises a multimodality sensor system that measures electrochemical, mechanical, structural, thermal, and/or energetic properties reflective of homeostasis and cell physiology. In some embodiments, the wearable device includes sensors that control and/or measure the tension within the tension mechanism. In some embodiments, the sensor may be connected to the band and/or the tension mechanism. In some embodiments, a first sensor may be based on circumferential measurement in which the sensor may dynamically measure the tension within the band and/or the tension within the tension mechanism. In some embodiments, a second sensor may dynamically measure various emergent factors of the biological compartment. For example, the second sensor may dynamically measure the pressure, heat flux, volume of the biological compartment, etc. In some embodiments, the sensor may measure the various emergent factors noninvasively or minimally invasively. In some embodiments, the sensor may dynamically measure the tension in the band and dynamically equate the tension to an internal pressure of the biological compartment. In some embodiments, the sensor may include an embedded microneedle to measure interstitial pressure minimally invasively dynamically. In some embodiments, the sensor may include a controller that may dynamically alter the tension in the tension mechanism and/or band based on the measurements measured by the sensor.

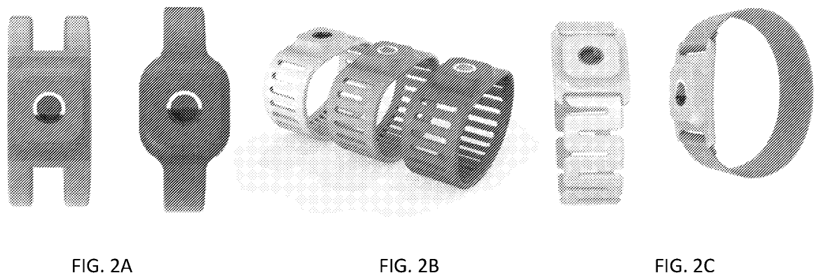

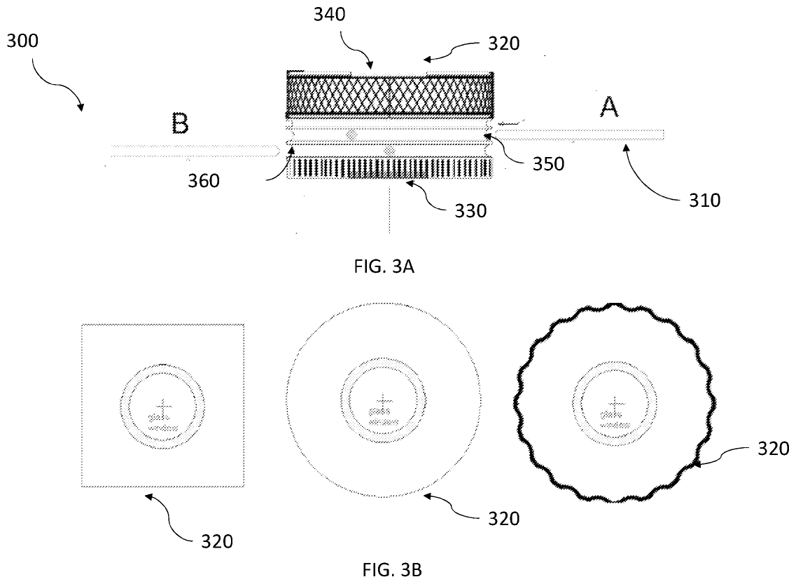

As shown in FIG. 3A, the wearable device 300 may include a band 310, a tension mechanism 320, a first sensor 330, and a second sensor 340. FIG. 3B shows the tension mechanism 320 can be square, round, or include a knurled finish. The tension mechanism 320 can be mechanically or automatically controlled. The first sensor 330 may dynamically measure various emergent factors of the user. The second sensor 340 may measure and/or control the tension within the tension mechanism 320. The first sensor 330 may include a thermal disc that may be flush to the user's skin during wear of the wearable device 300.

The tension mechanism 320 is designed to receive the band and may include a first channel 350 and a second channel 360. Depending on whether the tension mechanism 320 is to be tightened or to be loosened, the amount of the band 310 received by the first channel 350 and second channel 360 is designed so that it can increase or decrease. The tension mechanism 320 may use click frictional rotation feedback to maintain the band 310 and tension mechanism 320 at a desired tension. Each click frictional rotation can either take up the band 310 by wrapping the band 310 further around the first channel 350 and second channel 360 or let out the band 310 by unwrapping the band 310 from the channels. The band 310 may be similar to a chord bracelet. The material of the band 310 may be biocompatible silicone spaghetti chord of varying diameter for enhanced user comfort. The band 310 may have a diameter of approximately 0.5 mm to about 2 mm.

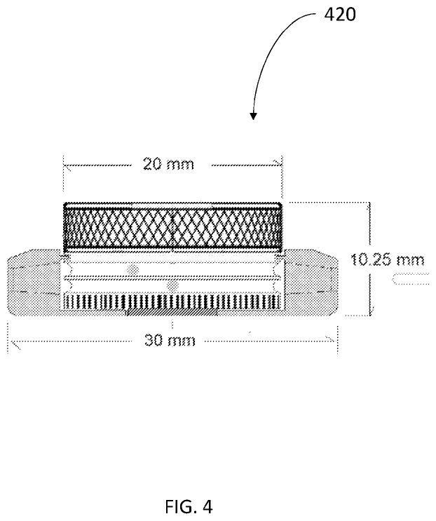

As shown in FIG. 4, the diameter of the tension mechanism 420 can be approximately 10 mm to about 30 mm. The diameter of the tension mechanism 420 may be proportional to the length of the band and range of adjustments to the length of the band permitted. The height of tension mechanism 420 can be approximately 8 mm to about 15 mm.

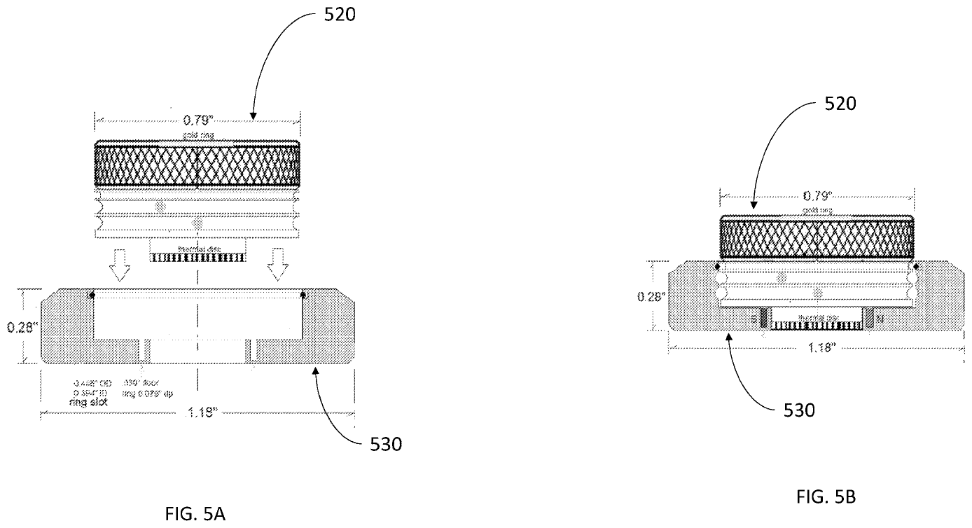

As shown in FIG. 5A-5B, the tension mechanism 520 may insert into a ring slot 530. The width of the ring slot 530 may range approximately between 1″ to about 2″. The height of ring slot 530 may range approximately between 0.1″ to about 0.4″. The diameter of the tension mechanism 520 may be approximately 0.5″-1.5″ and insert into an opening of the ring slot 520. As shown in FIG. 5A-5B, the opening of the ring slot may extend from the top of the ring slot 520 to the bottom of the ring slot 520. The diameter of the opening at the top of the ring slot 520 may be greater than the diameter of the opening at the bottom of the ring slot 520. In other words, the diameter of the opening may decrease throughout the depth of the ring slot. The diameter of the opening of the ring slot may be consistent throughout a portion of the ring slot and create a shelf upon the opening decreasing in diameter. The shelf allows for the tension mechanism to 520 to sit on the ring slot. The opening at the bottom of the ring slot 520 allows for the sensor to come in contact with the user's skin.

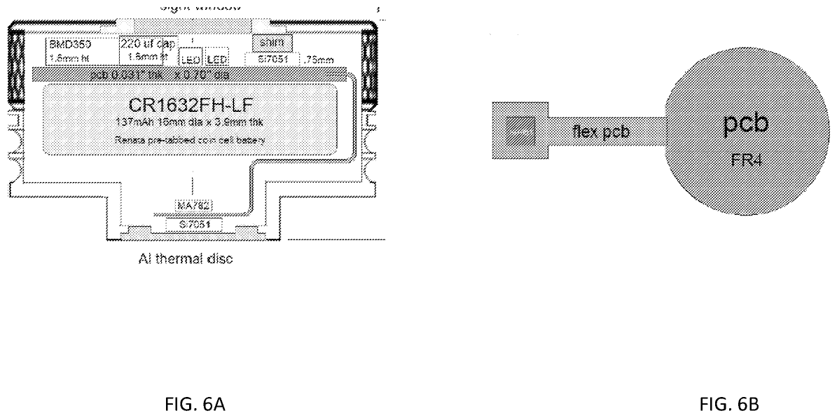

As shown in FIG. 6, a scale fit study demonstrates the components of the tension mechanism fit within an internal compartment of the tension mechanism.

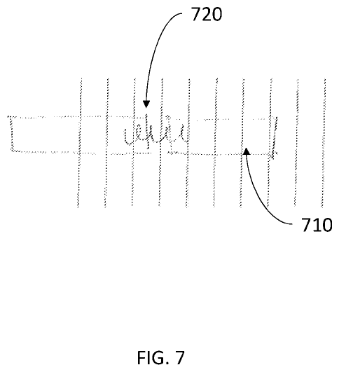

As shown in FIG. 7, the band 710 may include a spring 720. The spring 720 may allow the band 710 to increase in length to accommodate the biological compartment of the user.

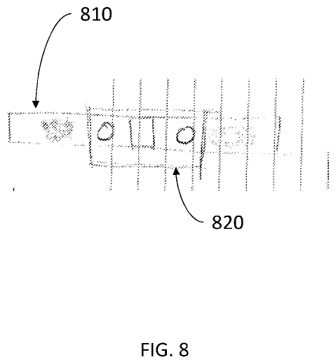

As shown in FIG. 8, the wearable device may include a band 810 and a clasp 820. The clasp 820 can be connected to the band 810. The clasp 820 can also connect a first end of the band 810 to a second end of the band 810. thereby creating a closed loop or a bracelet for the user to wear. The tension mechanism can be located within the clasp.

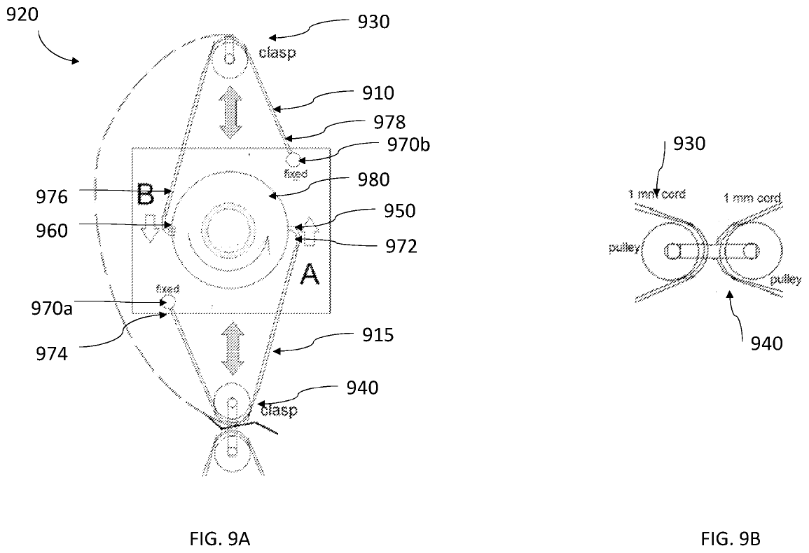

As shown in FIG. 9A-9B, a tension mechanism 920 includes at least two clasps 930, 940. The clasps 930, 940 can be a pulley system that assists the in the tightening or loosening of the wearable device. The pulley may include a channel that receives a middle portion of the band. As shown in FIG. 9A-9B, the first pulley 930 has a first channel that receives a first middle portion of a first portion of the band 910. A second pulley 940 includes a second channel that receives a second middle portion of a second portion of the band 915. The first and second middle portion of the band may be positioned within the first and second channel of the pulleys, respectively. The tension mechanism 920 may include a first connection point 950 and a second connection point 960. As shown in FIG. 9A, the first connection point 950 is positioned on a first side of the tension mechanism 920 and the second connection point 960 is positioned on a second side of the tension mechanism 920. The connection points serve to connect the band to the tension mechanism 920. The tension mechanism 920 further includes anchor points 970a, 970b to stabilize the band within the tension mechanism. As shown in FIG. 9A, a first end of the first portion of the band 972 connects to the first connection point 950 and a second end of the first portion of the band 974 connects to a first anchor point 970a located on the opposite side of the tension mechanism of the first connection point 950. Similarly, a first end of the second portion of the band 976 connects to the second connection point 960 and a second end of the second portion of the band 978 connects to the second anchor point 970b located on the opposite side of the tension mechanism of the second connection point 960. The tension mechanism further includes a knob 980 that is rotated to tighten or loosen the band. This may cause the band to increase or decrease in circumference. As shown in FIG. 9B, the clasps 930, 940 connect to secure the wearable device around the user's biological compartment.

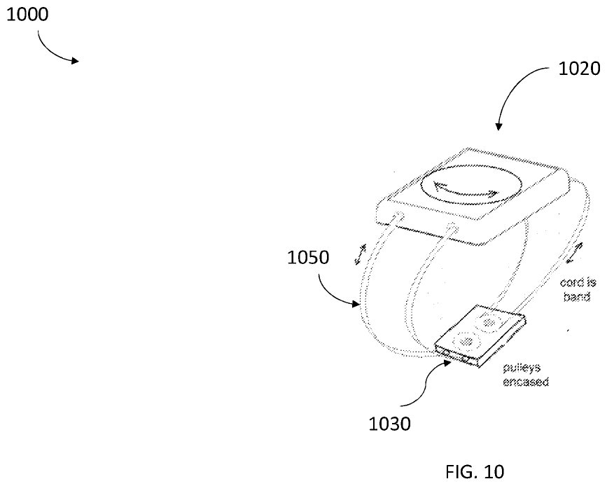

As shown in FIG. 10, the tension mechanism includes a first portion 1020 and a second portion 1030 that are parallel to each other during use of the wearable device 1000. The first portion 1020 and the second portion 1030 remain connected by a band 1050 that varies in length based on the variable size of user's biological compartment.

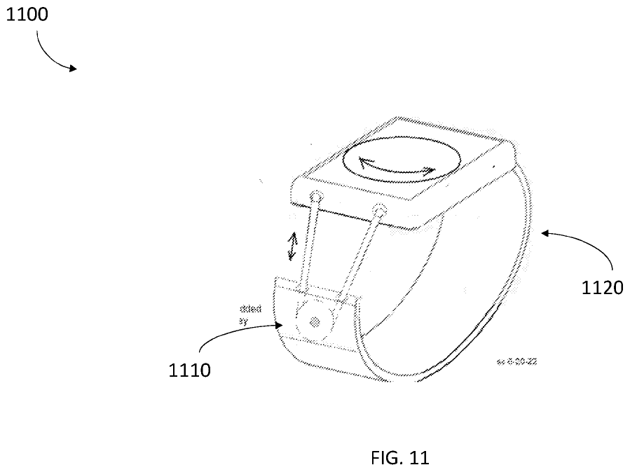

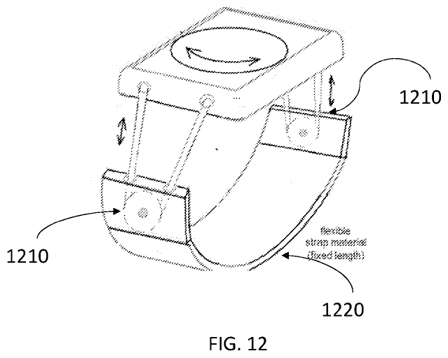

FIG. 11-12 show different ways in which the pulley system can be used within the tension mechanism. As shown in FIG. 11, the tension mechanism has a single pulley 1110 to dynamically adjust the size of the wearable device 1100. The wearable device 1100 includes a strap 1120 with the embedded pulley 1110 to enhance comfort for the portion of the wearable device that comes into contact with the user's skin. The strap can be a flexible material to further increase the ability of the wearable device to adjust to the varying size of the user's biological compartment. The flexible material of the strap can increase or decrease the size of the wearable device by approximately 1″ to about 4″. As shown in FIG. 12, the tension mechanism includes two pulleys 1210 embedded within a strap 1220, which can increase or decrease the size of the wearable device by approximately 1″ to about 6″.

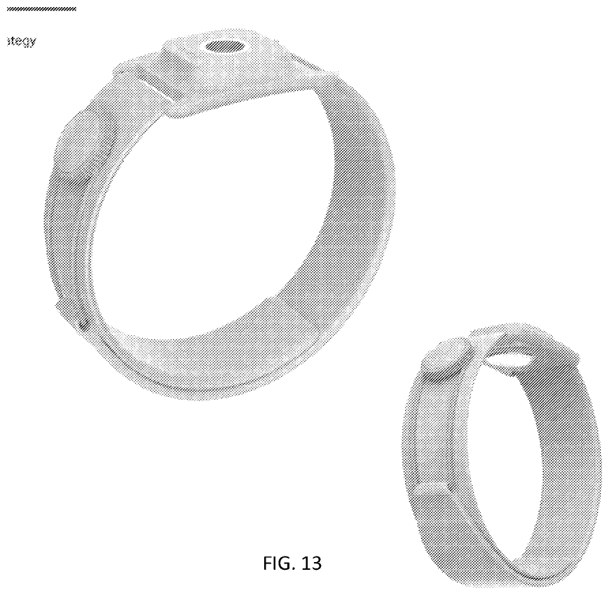

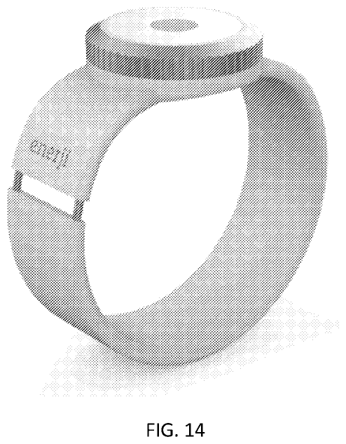

FIG. 13-17 show various wearable devices with the tension mechanisms described above. As shown in FIG. 13, the tension mechanism is on the external surface of the wearable device. This embodiment provides user easy access to the tension mechanism. As shown in FIG. 14, the tension mechanism is located within the internal compartment of the wearable device. This embodiment protects the tension mechanism from accidental adjustments.

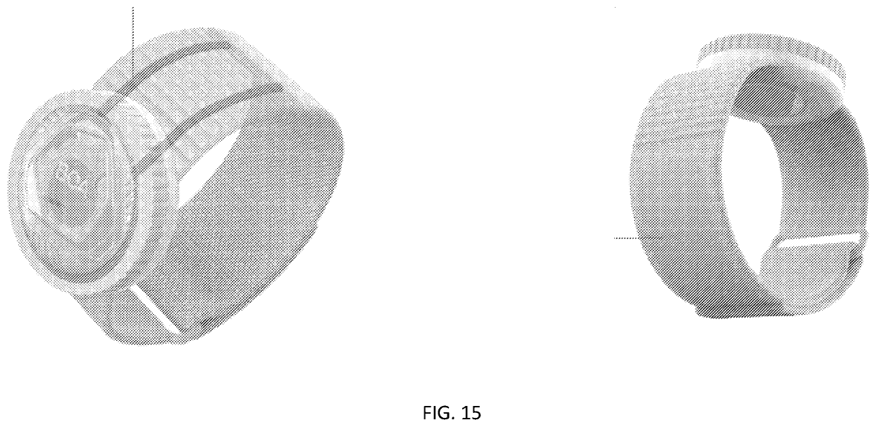

As shown in FIG. 15, the tension mechanism is also inside the wearable device. This embodiment allows for macro and micro adjustments of the size of the wearable device. As described above, the macro adjustments can be controlled by the length at which the user connects the first portion of the strap and the second portion of the strap. The micro adjustments can be controlled by the tension mechanism. The band of the tension mechanism connects to the strap to allow for the macro and micro adjustments to both increase or decrease the size of the wearable device. In other words, the macro and micro adjustments complement each other to ultimately maximize the user's comfort and prolong the wear of the wearable device.

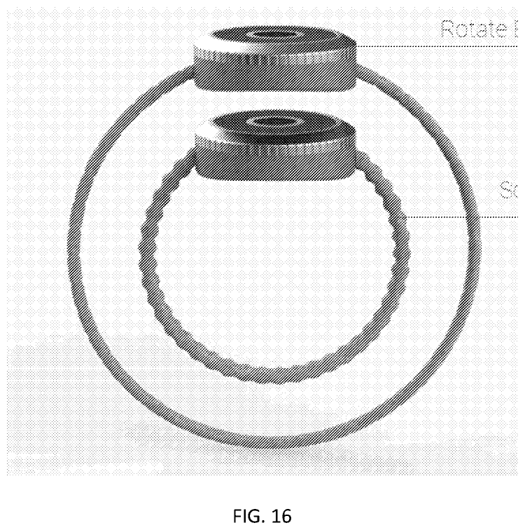

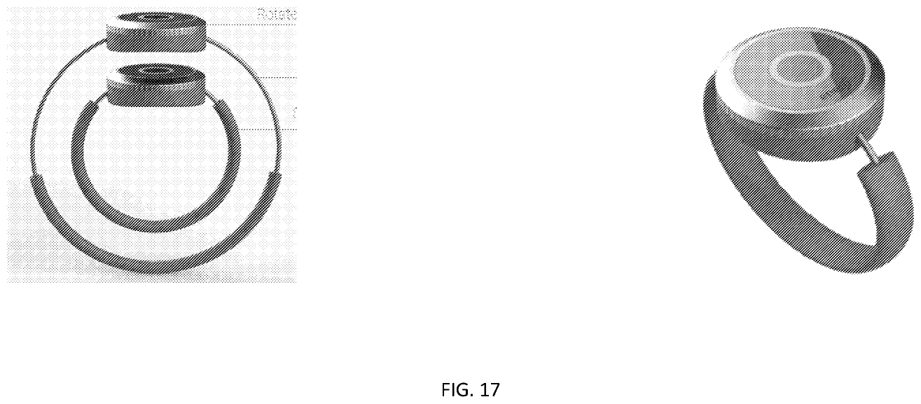

As shown in FIG. 16, a flexible material, or scrunchy like fabric, may encase or flex over the band of the tension mechanism. As shown in FIG. 17, the band of the tension mechanism may be revealed to decrease the overall weight of the wearable device.

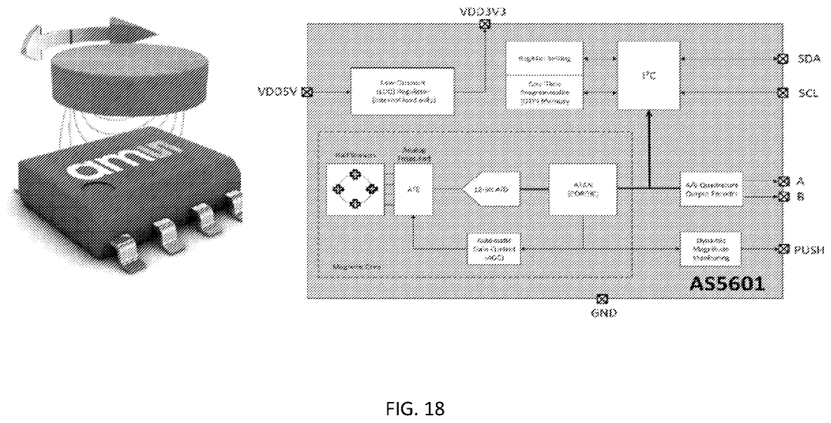

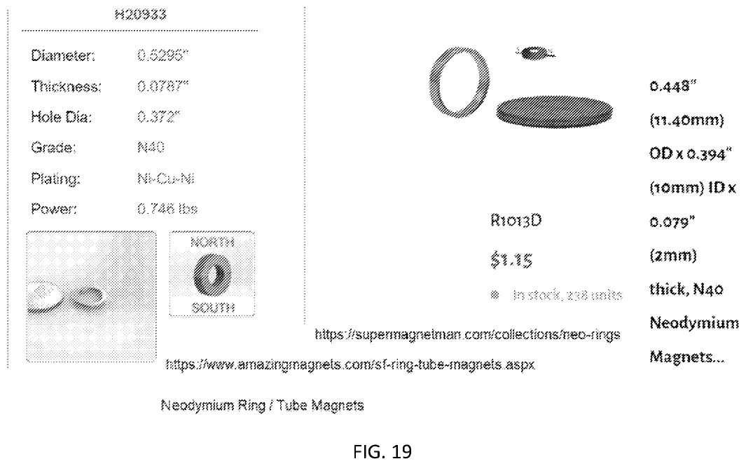

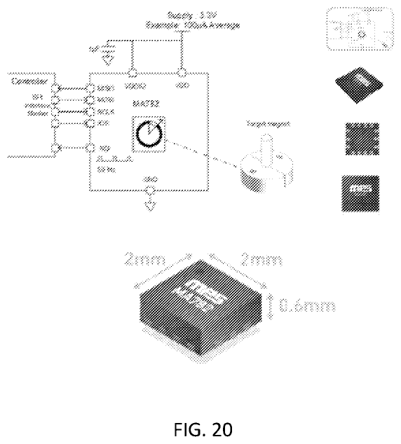

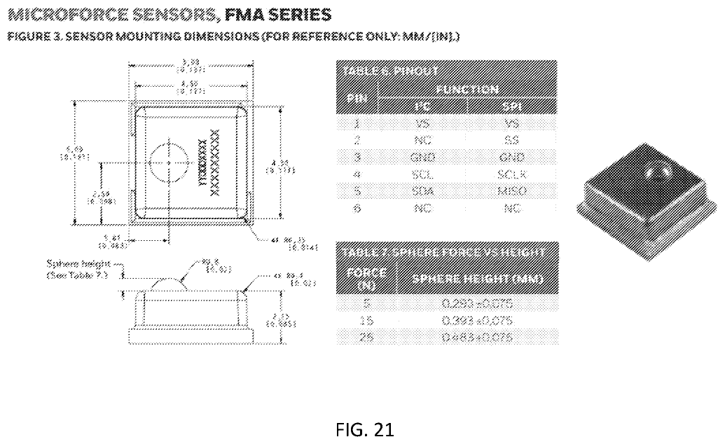

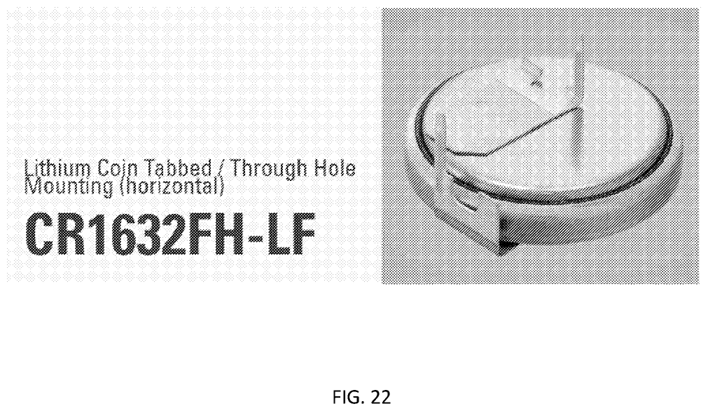

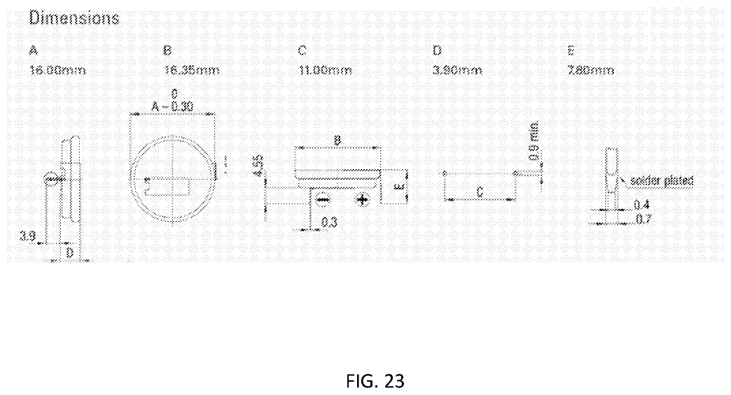

FIGS. 18-25 show the sensors used within the tension mechanism. As shown in FIG. 18-21, the sensor is used to transduce a signal control of the tension mechanism. This sensor controls the tightening and loosening of the tension mechanism and the overall size of the wearable device. The sensor includes an embedded magnet for the rotational signal to arise from a magnetic field orientation. The magnet may be a ceramic or ferrite donut magnet with a cross field orientation. FIGS. 19-20 demonstrate examples of magnets on the market. As shown in FIGS. 22-23, a battery' can be included within the tension mechanism to increase the use of the wearable device.

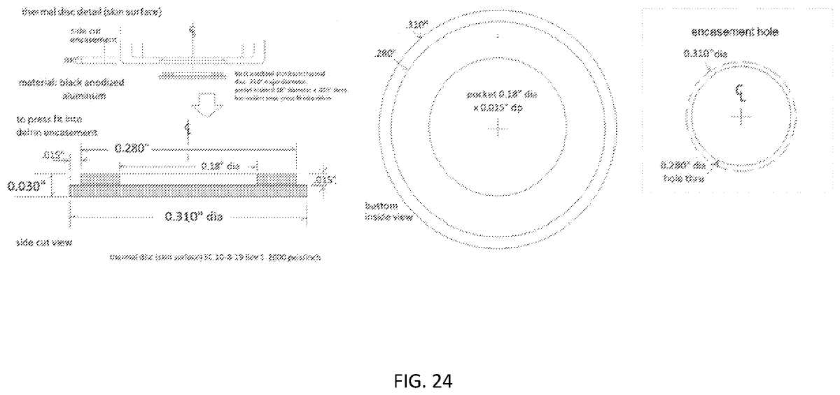

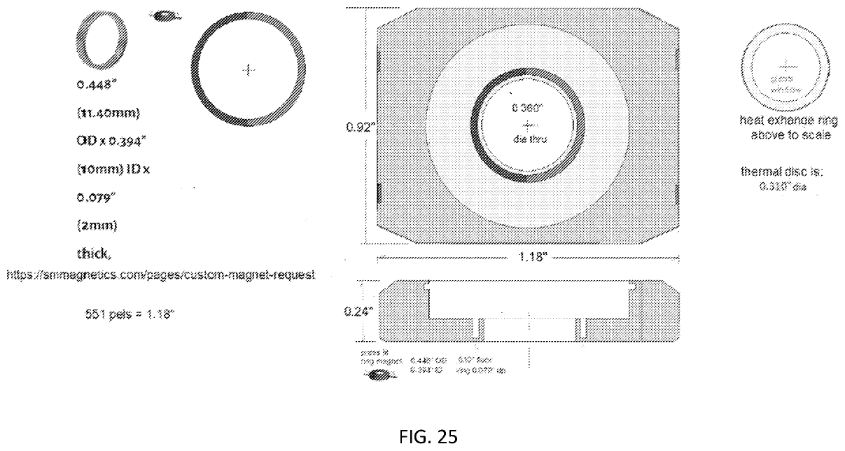

As shown in FIG. 24-25. the sensor is used to measure any number of emergent factors of the user. The sensor includes a thermal disc that can fit inside the ring magnet as described above. The thermal disc contacts the user's skin when the user is wearing the wearable device.

The embodiments described are examples. Various changes could be made in the above devices and methods without departing from the scope of the invention. All subject matter described in this disclosure, including the accompanying figures, is illustrative and not limiting.

### REFERENCES

Reference 1: Osilla E V, Marsidi J L, Sharma S. Physiology, Temperature Regulation. [Updated 2022 May 8], In: StatPearls [Internet], Treasure Island (FL): StatPearls Publishing; 2022 January-. Available from: https://www.ncbi.nlm.nih.gov/books/NBK507838/.
Reference 2: Schieber A M, Ayres J S. Thermoregulation as a disease tolerance defense strategy. Pathog Dis. 2016 December; 74(9): ftw106. doi: 10.1093/femspd/ftw106. Epub 2016 Nov. 3. PMID: 27815313; PMCID: PMC5975229.
Reference 3: Drewry A M, Fuller B M, Bailey T C, Hotchkiss R S. Body temperature patterns as a predictor of hospital-acquired sepsis in afebnle adult intensive care unit patients: a case-control study. Crit Care. 2013 Sep. 12; 17(5): R200. doi: 10. 1186/cc12894. PMID: 24028682; PMCID: PMC3906745.
Reference 4: Kenny G P, Sigal R J, McGinn R. Body temperature regulation in diabetes. Temperature (Austin). 2016 Jan. 4: 3(1): 119-45. doi: 10.1080/23328940.2015.1 131506. PMID: 27227101; PMCID: PMC4861190.
Reference 5: van Lier D, Geven C, Leijte G P, Pickkers P. Experimental human endotoxemia as a model of systemic inflammation. Biochimie. 2019 April; 159:99-106. doi: 10.1016/j.biochi.2018.06.014. Epub 2018 Jun. 22. PMID: 29936295.
Reference 6: Romanovsky A A. Skin temperature: its role in thermoregulation. Acta Physiol (Oxf). 2014 March; 210(3): 498-507. doi: 10. 1111/apha.12231. PMID: 24716231; PMCID: PMC4159593.
Reference 7: Altered Experienced Thermoregulation in Depression—No Evidence for an Effect of Early Life Stress—PubMed (nih.gov). Front Psychiatry. 2021 Jul. 21; 12:620656. doi: 10.3389/fpsyt.2021.620656. PMID: 34366905; PMCID: PMC8333702.
Reference 8: Hanusch, K., Janssen, C. H., Billheimer, D, Jenkins, I., Spurgeon, E., Lowry. C. A., Raison, C. L. Whole Body Hyperthermia (WBH) for the Treatment of Major Depression: Associations with Thermoregulatory Cooling. American Journal of Psychiatry 2013; 170:802-804.
Reference 9: Byrne J, Ludington-Hoe S M, Voss J G. Occupational Heat Stress, Thermal Comfort, and Cognitive Performance in the OR: An Integrative Review. AORN J. 2020 May; 1 11(5): 536-545. doi: 10. 1002/aom. 13009. PMID: 32343372.
Reference 10: Hancock, Peter A. and loannis Vasmatzidis. “Effects of heat stress on cognitive performance: the current state of knowledge.” International Journal of Hyperthermia 19 (2003): 355-372.

1. A device that is wearable by a user, comprising:
a band configured to be worn in substantially complete cross-sectional contact with a biological compartment of the user;
a tension mechanism connected to the band, the tension mechanism configured to allow a circumference of the band (i) to expand to accommodate ingress of fluid into the biological compartment and (ii) to contract to accommodate egress of fluid out of the biological compartment, such that the band maintains substantially complete cross-sectional contact with the biological compartment of the user; and
a sensor connected to the band, the sensor configured to collect a plurality of patient data.

2. The device of claim 1, wherein the tension mechanism is directly connected to the sensor.

3. The device of claim 1, wherein the tension mechanism is directly connected to the band.

4. The device of claim 1, wherein the tension mechanism is located within a clasp that is directly connected to the band.

5. The device of claim 1, wherein the tension mechanism includes a first mechanism and a second mechanism located in parallel when the band is in a closed position.

6. The device of claim 4, wherein the clasp is located in parallel with the sensor when the band is in a closed position.

7. The device of claim 1, wherein a first end of the band is configured to wind into a first band adaptor located within the tension mechanism, and a second end of the band is configured to wind into a second band adaptor located within the tension mechanism, wherein the first band adaptor and the second band adaptor configured to stabilize the first end of the band and the second end of the band.

8. The device of claim 1, wherein the tension mechanism comprises a spring.

9. The device of claim 8, wherein the spring is embedded within the band.

10. The device of claim 1, wherein the tension mechanism comprises a slider.

11. The device of claim 7, wherein the first end of the band overlaps with a second end of the band.

12. The device of claim 1, wherein the tension mechanism is round.

13. The device of claim 1, wherein the tension mechanism is knurled.

14. The device of claim 7, wherein the tension mechanism is configured to bring the first end of the band and the second end of the band together or apart.

15. The device of claim 4, wherein the clasp further includes at least two pulleys, the at least two pulleys configured to transmit tension from the band.

16. The device of claim 15, wherein a first connector is placed on a first pulley of the at least two pulleys and includes a first connector end and a second connector end, the first connector end is fixed, the second connector end is connected to the tension mechanism, the first connector is parallel to a second connector.

17. The device of claim 15, wherein a second connector is placed on a second pulley of the at least two pulleys and includes a first connector end and a second connector end, the first connector end is fixed, the second connector end is connected to the tension mechanism, the second connector is parallel to a first connector.

18. The device of claim 1, wherein the tension mechanism is configured to adjust based on a circumference of the biological compartment of the user.

19. The device of claim 18, wherein the tension mechanism is configured to adjust automatically based on the circumference of the biological compartment of the user.

20. The device of claim 1, wherein the biological compartment is at least one of a wrist and an ankle.

21. The device of claim 1, wherein the biological compartment is at least one of a finger and a toe.

22. The device of claim 1, wherein the biological compartment is at least one of a lower leg and a forearm.

23. The device of claim 1, wherein the biological compartment is at least one of a thigh and an upper arm.

24. The device of claim 1, wherein the biological compartment is an abdomen.

25. The device of claim 1, wherein the sensor is configured to dynamically measure pressure of the biological compartment.

26. The device of claim 1, wherein the sensor is configured to dynamically measure the circumference of the band.

27. The device of claim 1, wherein the sensor is configured to dynamically measure heat flux of the biological compartment.

28. The device of claim 1, wherein the sensor is configured to dynamically measure volume of the biological compartment.

29. The device of claim 1, comprising an additional sensor, wherein the additional sensor is configured to dynamically measure tension of the band.

30. The device of claim 1, comprising an additional sensor, wherein the additional sensor is configured to dynamically measure tension of the tension mechanism.

31-45. (canceled)

46. A method of assessing military readiness by utilizing the device of claim 1.
